(19) United States
(12) Patent Application Publication
(10) Pub. No.: US 2022/0354679 A1
(43) Pub. Date: Nov. 10, 2022

(54) STENT DELIVERY DEVICE AND GUIDE CATHETER

(71) Applicant: OLYMPUS CORPORATION, TOKYO (JP)

(72) Inventors: Hiromichi MIYANO, Fujisawa-shi (JP); Mayo KAMAGATA, Machida-shi (JP)

(73) Assignee: OLYMPUS CORPORATION, TOKYO (JP)

(21) Appl. No.: 17/872,114

(22) Filed: Jul. 25, 2022

Related U.S. Application Data
(63) Continuation of application No. PCT/JP2020/003199, filed on Jan. 29, 2020.

(51) Int. Cl. A61F 2/966; A61M 25/00

(52) U.S. Cl. CPC A61F 2/966; A61M 25/0043

(57) ABSTRACT

A catheter includes a first region having a first outer diameter; a second region having a second outer diameter larger than the first outer diameter; and a transition region connecting with the first region and the second region, the transition region having an outer diameter being equal to or larger than the first outer diameter and equal to or smaller than the second outer diameter, wherein the outer diameter of the transition region gradually decreases from the second region toward the first region, and part of an outer circumferential surface of the transition region is formed in a shape having a step portion.

This application is a continuation application of PCT International Application No. PCT/JP2020/003199, filed Jan. 29, 2020. The content of the PCT International Application is incorporated herein by reference.

### TECHNICAL FIELD

The present disclosure relates to a stent delivery device and a guide catheter.

### BACKGROUND ART

A medical catheter is widely used.

Generally, the catheter has an elongated shape formed in a tubular shape and made of the synthetic resin. The catheter includes the catheter introduced into the body through a natural orifice such as the mouth, the catheter for the blood vessels, and the like.

There is a case in which only a distal-end portion of the catheter is thinned to increase the insertability of the catheter into the body. On the other hand, in order to increase the strength of the catheter, there is a case in which only a proximal-end portion of the catheter is thickened. In these cases, the catheter has multiple regions with different diameters.

The catheter disclosed in Japanese Unexamined Patent Application, First Publication No. 2004-24692 has a distal-end portion and a main body portion that is thicker than the distal-end portion. A tapered portion whose diameter changes is formed between the main body portion and the distal-end portion over the entire circumference.

In the catheter disclosed in Japanese Unexamined Patent Application, First Publication No. 2004-24692, the position where the catheter breaks when an external load becomes excessive (hereinafter, referring to as a “breaking position”) is difficult to stabilize, and the catheter may break at the distal-end portion with a small outer diameter.

On the other hand, a structure in which a boundary between the distal-end portion and the main body portion has only a step portion without providing the tapered portion is known. In this case, the breaking position is stable, however, the distal-end portion having the small outer diameter breaks such that the breaking strength is low.

### SUMMARY

According to an aspect of the present disclosure, a catheter includes a first region having a first outer diameter; a second region having a second outer diameter larger than the first outer diameter; and a transition region connecting with the first region and the second region, the transition region having an outer diameter being equal to or larger than the first outer diameter and equal to or smaller than the second outer diameter. The outer diameter of the transition region gradually decreases from the second region toward the first region, and part of an outer circumferential surface of the transition region is formed in a shape having a step portion.

According to another aspect of the present disclosure, a stent delivery device includes a delivery catheter including a tubular stent and a guide tube inserted into the stent. The guide tube includes a first region having a first outer diameter; a second region having a second outer diameter larger than the first outer diameter; and a transition region connecting with the first region and the second region, the transition region having an outer diameter being equal to or larger than the first outer diameter and equal to or smaller than the second outer diameter. The outer diameter of the transition region gradually decreases from the second region toward the first region, part of an outer circumferential surface of the transition region is formed in a shape having a step portion, and the step portion is positioned between a proximal end of the stent and the second region.

According to a further aspect of the present disclosure, a stent delivery device includes a delivery catheter including a tubular stent and a guide tube inserted into the stent. The guide tube includes a first region having a first outer diameter; a second region having a second outer diameter larger than the first outer diameter; and a transition region connecting with the first region and the second region, the transition region having an outer diameter being equal to or larger than the first outer diameter and equal to or smaller than the second outer diameter. A breaking strength of the transition region along a longitudinal direction of the guide tube is in a range from 45N to 80N.

### BRIEF DESCRIPTION OF DRAWINGS

FIG. 1 is an overall view showing a stent delivery device according to an embodiment of the present disclosure.

FIG. 2 is a side view showing a stent according to the stent delivery device.

FIG. 3 is a view showing an internal structure of the stent.

FIG. 4 is a schematic cross-sectional view of the stent delivery device.

FIG. 5 is a partial enlarged view showing a delivery catheter according to the stent delivery device.

FIG. 6 is a cross-sectional view along a line I-I in FIG. 5.

FIG. 7 is a cross-sectional view along a line II-II in FIG. 5.

FIG. 8 is a cross-sectional view along a line III-III in FIG. 7.

FIG. 9 is a cross-sectional view along a line IV-IV in FIG. 7.

FIG. 10 is a view showing a procedure in a manufacturing procedures of the delivery catheter.

FIG. 11 is a view showing a procedure in the manufacturing procedures of the delivery catheter.

FIG. 12 is a view showing a procedure in the manufacturing procedures of the delivery catheter.

FIG. 13 is a view showing a position of transition area during the usage of the stent delivery device.

FIG. 14 is a view showing a part of a conventional catheter.

FIG. 15 is a table showing experimental results on the difference between a shape of the tube and the breaking strength and the breaking position.

FIG. 16 is a partial enlarged view showing a delivery catheter according to a modification example of the present disclosure.

FIG. 17 is a partial view showing the delivery catheter according to the modification example of the present disclosure.

FIG. 18 is a partial enlarged view showing the delivery catheter according to the modification example of the present disclosure.

### DESCRIPTION OF EMBODIMENTS

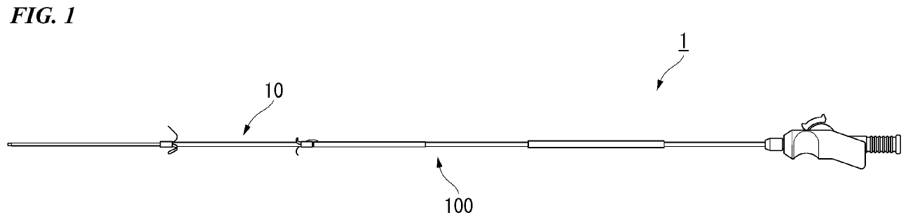

An embodiment of the present disclosure will be described with reference to FIG. 1 to FIG. 14.

FIG. 1 is an overall view of a stent delivery device 1 according to the present embodiment. The stent delivery device 1 includes a tubular stent 10 and a delivery catheter 100. The delivery catheter 100 is the catheter according to the present disclosure.

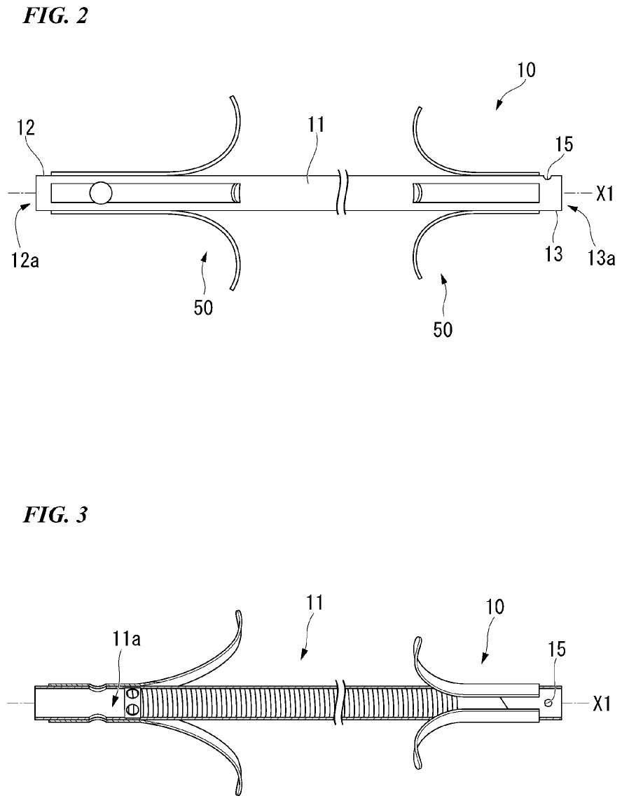

FIG. 2 is a side view of the stent 10. The stent 10 according to the present embodiment is a stent disposed in the bile duct, and the stent 10 includes a tubular main body 11 and flaps 50 attached to both ends of the main body 11. The main body 11 has a distal end 12 having a distal-end opening 12a and a proximal end 13 having a proximal-end opening 13a, and the main body 11 extends along a longitudinal axis X1. A stent lumen 11a extends along the longitudinal axis X1 between the distal-end opening 12a and the proximal-end opening 13a. The distal end 12 is an end portion that is disposed on the liver side when the stent 10 is indwelled in the bile duct. The proximal end 13 is an end portion that is disposed on the duodenal papilla side when the stent 10 is indwelled in the bile duct.

FIG. 3 is a view showing an internal structure of the stent 10. A hole 15 communicating with the stent lumen 11a is formed on an outer circumferential surface of one end portion of the stent 10. The hole 15 is used to temporarily connect the stent 10 and the delivery catheter 100, as will be described later.

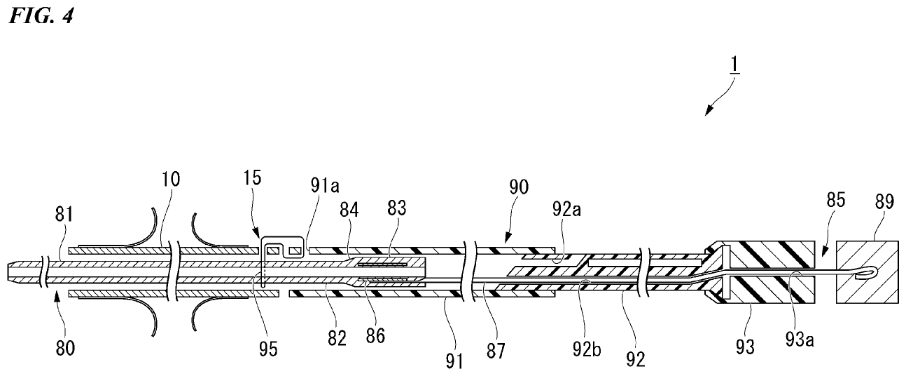

FIG. 4 is a schematic cross-sectional view showing the structure of the stent delivery device 1. The delivery catheter 100 includes a guide catheter (guide tube) 80 and a pusher catheter 90.

The guide catheter 80 has a tube 81 through which a guide wire can be inserted and a traction portion 85 for moving the tube 81.

The tube 81 is a tubular member made of resin and has an internal space through which the guide wire can be inserted. A diameter (inner diameter) of the internal space is preferably the same in the longitudinal direction of the tube 81. The tube 81 is flexible enough to be deformed when the tube 81 comes into contact with the living tissue when the stent delivery device 1 is used. The tube 81 is an elastic member having a restoring force, and the tube 81 is in a linear shape due to the restoring force in a state where no external force is applied thereto. The tube 81 has a small-diameter portion (first region) 82 positioned on the distal-end side of the stent delivery device 1 and a large-diameter portion (second region) 83 positioned on the proximal-end side of the stent delivery device 1 and having an outer diameter larger than that of the small-diameter portion 82. Between the small-diameter portion 82 and the large-diameter portion 83, a transition region 84 in which an outer diameter (diameter-direction dimension) thereof gradually decreases from the large-diameter portion 83 toward the small-diameter portion 82 in the longitudinal axis direction is formed, and the small-diameter portion 82 and the large-diameter portion 83 are connected by the transition region 84. The outer diameter of the transition region 84 varies depending on the portion of the transition region 84, however, the outer diameter at any portion thereof is equal to or larger than the outer diameter of the small-diameter portion 82 (first outer diameter) and equal to or smaller than the outer diameter of the large-diameter portion 83 (second outer diameter). Details will be described later.

Since the first outer diameter is smaller than the inner diameter of the stent 10, the transition region 84 can be inserted into the inside of the stent. Therefore, the stent 10 is attached to the delivery system by inserting the small-diameter portion (first region) 82 as a part of the guide catheter 80 inside of the stent 10. Further, in the state in which the stent 10 is attached to the delivery catheter 100, the transition region 84 and the large-diameter portion 83 are positioned in the pusher catheter 90 on the hand side, and a part of the small-diameter portion 82 is inserted into the stent 10 such that the distal-end portion of the small-diameter portion 82 protrudes from the distal end of the stent 10.

The longitudinal axis X of the tube 81 substantially coincides with the central axis of the delivery catheter 100.

A material of the tube 81 is the fluororesin, the thermoplastic resin, or the like, and the material can be shown as the following examples. The material is not particularly limited as long as the desired mechanical properties of the tube 81 are satisfied.

The examples of the material of the tube 81 may be the olefin resins such as the polypropylene, the polyethylene, and their copolymer resins, the polyester resins such as the polyethylene terephthalate (PET) and the polybutylene terephthalate (PBT), and the general-purpose resins such as the polyvinyl alcohol (PVA).

The examples of the material of the tube 81 may be the engineering resins such as polyamide-based resins, fluorine-based resins (for example, the polytetrafluoroethylene (PTFE), the polyvinylidene fluoride (PVDF), PFA, FEP, ETFE, etc.), the polyetheretherketone (PEEK), and the like.

In addition, the examples of the material of the tube 81 may be various elastomer resins (polystyrene-based, polyolefin-based, polyurethane-based, polyester-based, polyamide-based, polyvinyl chloride-based, etc.), the silicone-containing resins, the polyurethane-based resins, and the like.

The traction portion 85 includes a pipe 86, a wire 87, and an operation portion 89. The pipe 86 is a metal tubular member having openings at both ends open in the axial direction. The pipe 86 is mounted to the inside of the tube 81 coaxially with the tube 81. The pipe 86 is embedded in the wall of the large-diameter portion 83.

Examples of the material of the pipe 86 include a metal such as the stainless steel or the like, and the engineering resin such as the PEEK; however, other materials may be used as long as the desired mechanical properties are satisfied.

The distal-end portion of the wire 87 is joined to the pipe 86 by welding or the like, and the proximal-end portion is connected to the operation portion 89.

As the material of the wire 87, the same examples of the material forming the pipe 86 can be used. Other materials may be used as long as the desired mechanical properties.

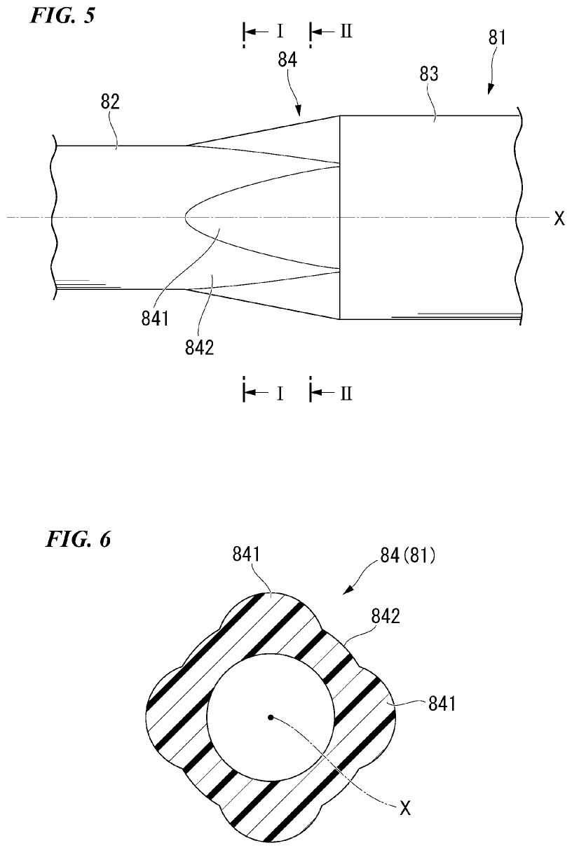

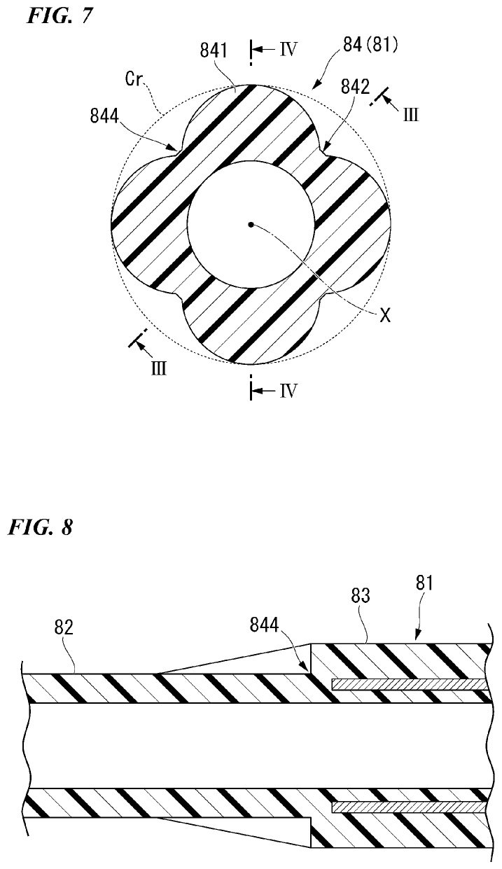

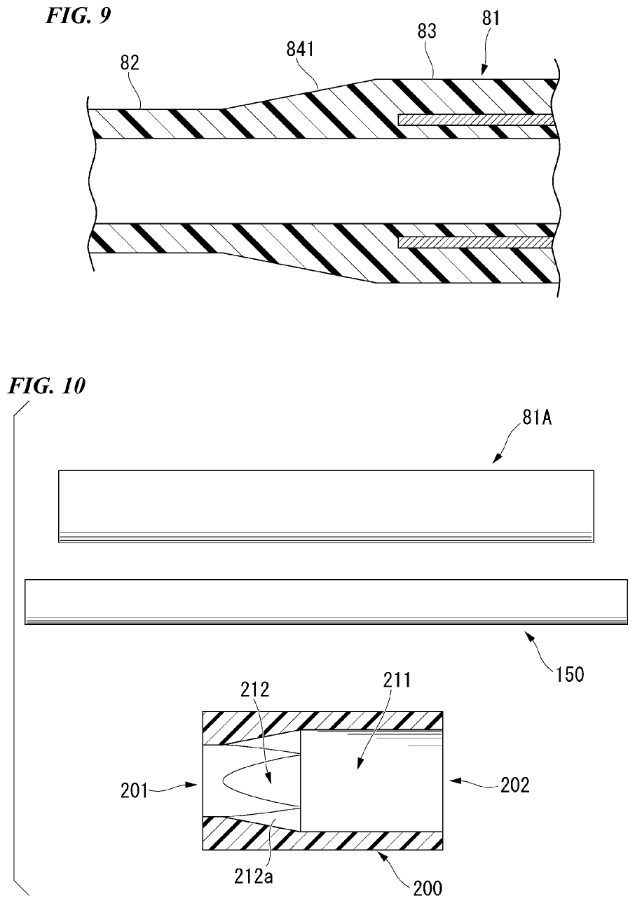

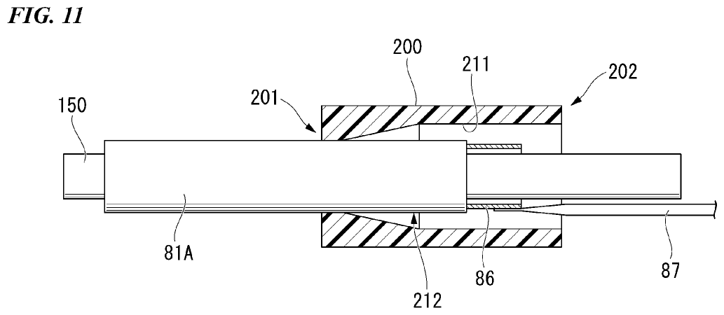

FIG. 5 is a view showing the transition region 84 and the vicinity of the transition region 84. FIG. 6 is a view showing a cross section taken along the line I-I in FIG. 5. FIG. 7 is a view showing a cross section taken along the line II-II in FIG. 5. FIG. 8 is a view showing a cross section taken along the line III-III in FIG. 7. FIG. 9 is a view showing a cross section taken along the line IV-IV in FIG. 7.

As shown in FIG. 5 to FIG. 7, the transition region 84 has four protrusions 841 arranged in the circumferential direction of the tube 81. The protrusions 841 have the same shape and the same size with each other, and the protrusions 841 are arranged at equal intervals in the circumferential direction of the tube 81. As shown in FIG. 9, as the small-diameter portion 82 approaches the large-diameter portion 83, the distance from the central axis in the cross section of the tube 81 to the apex of each protrusion 841 (the outer circumferential surface of the transition region 84) gradually increases, and the outer circumferential surface of the transition region 84 is continuous with the outer circumferential surface of the large-diameter portion 83. That is, the surface of each protrusion 841 is continuously connected between the outer circumferential surface of the small-diameter portion 82 and the outer circumferential surface of the large-diameter portion 83 without any step portion, and the outer diameter of the transition region 84 gradually decreases from the large-diameter portion 83 toward the small-diameter portion 82 by taking the large-diameter portion 83 as the reference.

The width of each protrusion 841 is the largest value at the proximal end of the transition region 84, and gradually decreases as approaching the distal end of the transition region 84.

As shown in FIG. 7 and FIG. 8, a part of the outer circumferential surface of the transition region 84 has a shape forming a step portion. Specifically, the transition region 84 has a plurality of protrusions 841 protruding from the outer circumferential surface of the small-diameter portion 82, and has steps 844 between the protrusions 841 respectively. As shown in FIG. 7, the step 844 is a recess portion 842 having a shape recessed from the protrusion 841 in the circumferential direction of the transition region 84, for example. The plurality of recess portions 842 are located at equal intervals in the circumferential direction of the transition region 84 and are continuous with the outer circumferential surface of the small-diameter portion 82, and a step 844 with respect to the outer circumferential surface of the large-diameter portion 83 is generated in the proximal end of the transition region 84. In a state in which the stent 10 is attached to the delivery catheter 100, the step 844 is positioned between the proximal end of the stent 10 and the large-diameter portion 83.

The recess portion according to the present disclosure means a portion positioned at the internal side of a circle Cr having the longitudinal axis X as a center and passing through a portion that is the farthest from the longitudinal axis X in the cross section of the transition region 84 orthogonal to the longitudinal axis X.

The manufacturing procedures of the guide catheter 80 will be described.

At first, as shown in FIG. 10, a material tube 81A having the same diameter as that of the small diameter portion 82, a core metal 150 passing through the material tube 81A, and a mold 200 having a cavity corresponding to the shape of the transition region 84 and the large-diameter portion 83 are prepared. The mold 200 has a first opening 201 corresponding to the small-diameter portion 82 and a second opening 202 corresponding to the large-diameter portion 83. The material of the mold 200 is not particularly limited, and the metal, the heat-resistant resin, or the like can be used.

The cavity of the mold 200 has a columnar-shaped first cavity 211 having an inner diameter corresponding to the outer diameter (second outer diameter) of the large-diameter portion 83, and a transition cavity 212 that communicates with the first cavity 211. The inner surface of the transition cavity 212 has a shape corresponding to the inner surface shape of the transition region 84, and has a plurality of convex portions 212a corresponding to the concave portions 842, respectively.

Next, as shown in FIG. 11, the material tube 81A through which the core metal 150 is passed is arranged in the mold 200 in a state of protruding from the first opening 201, and the pipe 86 to which the wire 87 is joined is inserted from the second opening 202 into the mold 200 and disposed inside the first cavity 211.

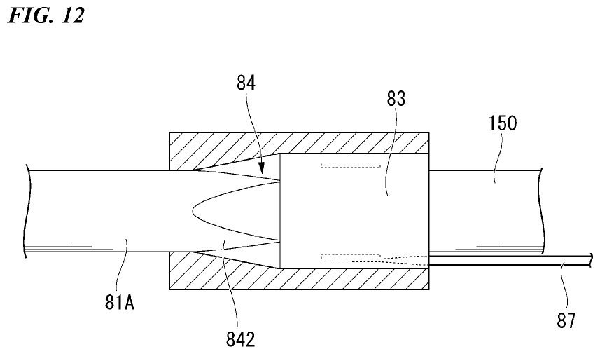

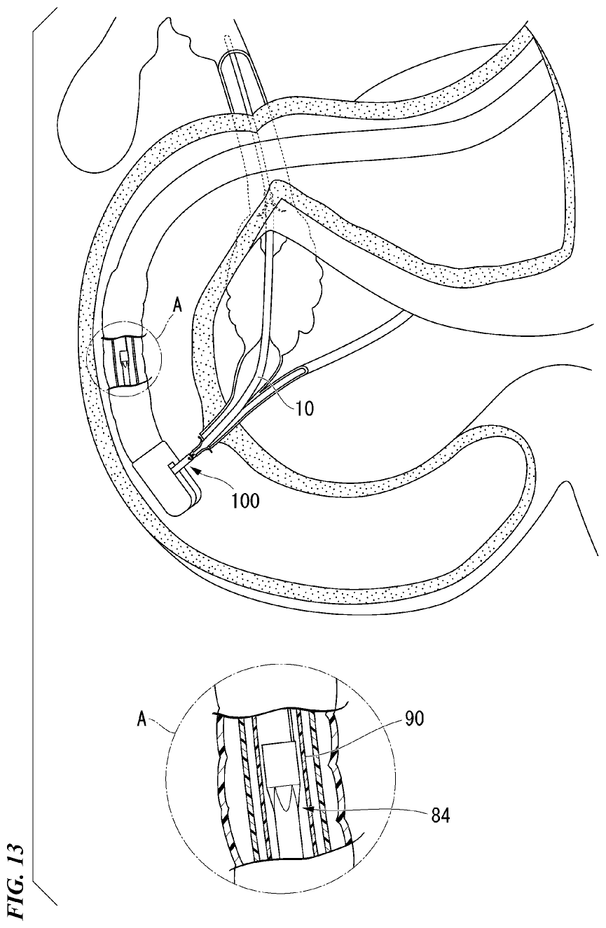

In this state, when the inside of the mold 200 is heated, for example, the material tube 81A melts, and as shown in FIG. 12, a part of the melted material tube 81A is molded into the shape of the large-diameter portion 83 corresponding to the first cavity 211 and the shape of the transition cavity 84 corresponding to the transition cavity 212. At this time, the pipe 86 is covered with the melted material tube 81A and embedded in the melted material tube 81A.

Next, when the heating in the mold 200 is stopped, the temperature of the material tube 81A melted in the mold 200 decreases.

After the transition region 84 and the large-diameter portion 83 are solidified, the molded material tube 81A is pulled out from the second opening 202 to complete the guide catheter 80 having the small-diameter portion 82, the large-diameter portion 83, and the transition region 84. The operation portion 89 may be attached to the wire 87 at an appropriate timing.

In the above description, an example of using one material tube has been described; however, another material tube having a diameter close to that of the large-diameter portion may be put in the mold 200, and the two material tubes may be connected in the mold 200 to form the tube 81.

As shown in FIG. 4, the pusher catheter 90 has a single lumen tube 91, a multi-lumen tube 92, and a grasp portion 93.

The single lumen tube 91 is a tubular member having an internal space into which the large-diameter portion 83 of the tube 81 can be inserted. The single lumen tube 91 has the flexibility. The distal-end surface of the single lumen tube 91 is a plane orthogonal to the center line of the single lumen tube 91. The distal-end surface of the single lumen tube 91 can abut against the proximal end of the stent 10 to support the stent 10. The size of the wall thickness of the single lumen tube 91 is equal to or larger than the difference between the inner radius and the outer radius of the main body 11 of the stent 10 (that is, the wall thickness of the stent 10). The single lumen tube 91 has a length so as to be able to completely accommodate the large-diameter portion 83 of the tube 81 inside the single lumen tube 91.

The multi-lumen tube 92 is fixed to the proximal-end portion of the single lumen tube 91. The multi-lumen tube 92 has a guide-wire lumen 92a for inserting the guide wire and a wire lumen 92b. The wire 87 of the guide catheter 80 is inserted through the wire lumen 92b.

The guide-wire lumen 92a is open at the distal end of the multi-lumen tube 92 while being open at the side surface of the multi-lumen tube 92 on the proximal end side with respect to the distal end of the multi-lumen tube 92.

The wire lumen 92b is open at the distal end and the proximal end of the multi-lumen tube 92.

The grasp portion 93 is connected to the proximal-end portion of the multi-lumen tube 92. The grasp portion 93 has a substantially cylindrical shape having a diameter larger than that of the multi-lumen tube 22. An unevenness portion or the like for slip-proof or the like may be formed on the outer circumferential surface of the grasp portion 93.

A through hole 93a that communicates with the wire lumen 92b is formed in the grasp portion 93. The through hole 93a is positioned on an extension line toward the proximal-end side of the center line of the wire lumen 92b. In a case in which the inner diameter of the through hole 93a is sufficiently large, it is not necessary for the through hole 93a to be positioned on the extension line of the center line of the wire lumen 92b.

The wire 87 of the guide catheter 80 is inserted through the through hole 93a. As a result, the wire 87 and the operation portion 89 extend outside from the through hole 93a.

As the single lumen tube 91 and the multi-lumen tube 92, it is preferable to use the same type of materials with different blending ratio only. In this case, when both are welded and joined, it is easy to adjust to the desired bending rigidity while maintaining the joining strength.

As the resin material of the single lumen tube 91 and the multi-lumen tube 92, the same resin as that of the tube 81 can be used. For example, when a relatively flexible elastomer resin and a relatively rigid thermoplastic resin are blended and the blending ratio of the thermoplastic resin in the multi-lumen tube 92 is higher than that of the single lumen tube 91, it is possible to make the bending rigidity of the single lumen tube 91 to be less than the bending rigidity of the multi-lumen tube 92 so as to improve the insertability of the delivery catheter 100.

The stent 10 is passed through the tube 81 protruding from the pusher catheter 90. The stent 10 is attached to the delivery catheter 100 in a state in which the end portion where the hole 15 is provided (see FIG. 2 and FIG. 4) is positioned on the pusher catheter 90 side.

A hole 91a communicating with the internal space is provided at the distal-end portion of the single lumen tube 91 as shown in FIG. 4. A thread 95 is passed through the hole 91a. Since the thread 95 is in a shape in which both ends thereof are tied to form a loop shape in a state of passing through the hole 91a, the thread 95 is supported by the single lumen tube 91 so as to not to slip off from the hole 91a.

The loop-shaped thread 95 has entered the stent 10 through the hole 15. The tube 81 passes through the loop shape of the thread 95 in the stent 10. Therefore, the thread 95 does not slip out of the hole 15 unless the tube 81 slips out of the loop of the thread 95. Accordingly, by passing the tube 81 through the loop of the thread 95, the stent 10 is temporarily attached to the pusher catheter 90 via the thread 95. By pulling the tube 81 out of the loop of the thread 95, the thread 95 is disengaged from the stent 10 such that the stent 10 is released from the pusher catheter 90.

The nylon can be shown as an example of the material of the thread 95.

The dimensional examples of each portion of the delivery catheter 100 are shown as follows; however, the configuration according to the present embodiment is not limited to these examples.

Overall length of the guide catheter 80: 2100 mm to 2300 mm

Length of the tube 81: 350 mm to 450 mm

Length of the wire 87: 1750 mm to 1850 mm

Overall length of the pusher catheter 90: 1700 mm to 1800 mm

Overall length of the single lumen tube 91: 480 mm to 520 mm

Overall length of the multi-lumen tube 92: 1220 mm to 1280 mm

The operations of the stent delivery device 1 configured as described above will be described using an example in a state in which the stent 10 is indwelled in the bile duct.

A surgeon passes the guide wire through a channel of a side-view endoscope and inserts the guide wire into the bile duct while observing with the endoscope. Subsequently, the surgeon operates the guide wire under the fluoroscopy to break through the stenotic site in the bile duct and move the distal-end portion of the guide wire closer to the liver side with respect to the stenotic site.

The surgeon inserts the proximal-end portion of the guide wire protruding from the forceps port of the endoscope into the distal-end opening of the tube 81 of the stent delivery device 1 to which the stent 10 is attached. The guide wire enters the lumen of the single lumen tube 91 through the proximal-end opening of the tube 81. Further, the surgeon makes the proximal-end portion of the guide wire to enter the guide-wire lumen 92a and protrudes the proximal-end portion of the guide wire from the proximal-end side opening (side opening) of the guide-wire lumen 92a.

The surgeon inserts the stent delivery device 1 through which the guide wire is passed into the channel of the endoscope, and projects the distal-end portion of the stent delivery device 1 from the distal end of the channel. The surgeon operates a raising base of the endoscope to direct the distal end of the stent delivery device 1 toward the duodenal papilla and make the stent delivery device 1 to enter the bile duct along the guide wire. In a state in which the stent 10 is temporarily connected to the pusher catheter 90 by the thread 95, the stent 10 is arranged on the distal-end side of the guide catheter 80 with respect to the large-diameter portion 83 of the tube 81, and the transition region 84 is positioned inside the single-lumen tube 91.

When the distal-end portion of the stent 10 breaks through the stenosis site and the flaps 50 at the distal-end side move to the liver side with respect to the stenosis site, the surgeon advances and retracts the stent delivery device 1 to determine the indwelling position of the stent 10. In the stent delivery device 1, since the stent 10 and the delivery catheter 100 are temporarily connected as described above, it is possible to retract the stent 10 by retracting the stent delivery device 1. Therefore, the position of the stent 10 can be easily adjusted.

As shown in FIG. 13, the transition region 84 is positioned at the hand side with respect to the proximal end of the stent 10. Specifically, in a state in which the stent 10 is attached to the delivery catheter 100, the transition region 84 is positioned in the pusher catheter 90 at the hand side with respect to the stent 10.

In FIG. 13, the range A on the upper side is enlarged to be shown on the lower side.

After the indwelling position of the stent 10 is determined, the surgeon pulls the operation portion 89 toward the hand side while holding the pusher catheter 90. Then, the wire 87 and the tube 81 retract; however, the stent 10 is in contact with the pusher catheter 90 and does not retract. When the tube 81 retracts and slips out of the loop of the stent 10 and the thread 95, the thread comes off the stent 10 and the stent 10 is released from the pusher catheter 90. When the tube 81 is further retracted, the stent 10 disengages from the guide catheter 80 and the stent 10 is indwelled in a desired position within the bile duct.

In a case in which the cavity of the stenosis site St is narrow or the like, there is a case in which the stent 10 passing through the stenosis site St may be strongly pressed against the inner wall of the stenosis site St and strongly pressed against the tube 81. In this case, even if the operation portion 89 is pulled toward the hand side, the tube 81 does not retract and a large force is applied on the guide catheter 80. Since the outer circumferential surface of the transition region 84 has a shape that partially forms the step with respect to the outer circumferential surface of the large-diameter portion 83, it is easy for the stress to be concentrated at the boundary between the transition region 84 and the large-diameter portion 83. Therefore, even if the entire stent delivery device 1 is pulled too strongly toward the hand side in the state in which the stent 10 and the tube 81 cannot move relative to each other due to the stenosis site St, the tube 81 does not separate into two portions at the position being difficult for the snare or the grasping forceps to approach such as inside the stent 10 or the like.

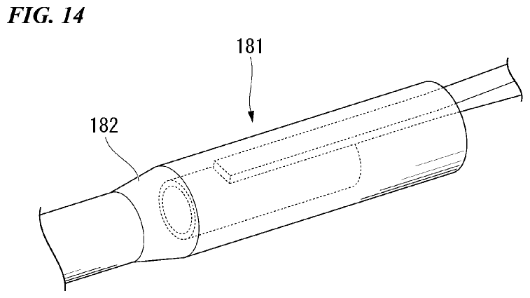

According to the present embodiment, a step shape is partially formed at the boundary between the transition region 84 and the large-diameter portion 83 by partially providing the recess portion 842 in the transition region 84. Therefore, according to the present disclosure, as compared with a case of forming a conventional catheter 181 only having the truncated-cone-shaped tapered portion 182 as shown in FIG. 14 by the same procedure, it becomes difficult to break at the small-diameter portion 82 while it becomes easy to break at the position where the partial step portion is formed.

On the other hand, in a structure in which only the step portion is formed between the small-diameter portion 82 and the large-diameter portion 84 (the comparison example described later), the small diameter portion breaks. When the recess portion 842 is partially provided in the transition region 84, the cross-sectional area of the transition region 84 becomes larger than the cross-sectional area of the small-diameter portion 82, such that the breaking strength can be made higher than that of the comparison example. Furthermore, in the case when the catheter breaks, it is almost definitely that the catheter breaks at the transition region 84.

As described above, the delivery catheter 100 according to the present embodiment, the breaking position thereof is stabilized at the portion where the breaking strength becomes high.

The experimental results on the difference between the breaking strength and the breaking position due to the structure of the boundary region between the small-diameter portion and the large-diameter portion will be described.

Three types of tubes were manufactured under the same conditions using the same material tubes and molds having different cavity shapes. In each example, the dimensions of the large diameter portion and the small diameter portion were the same.

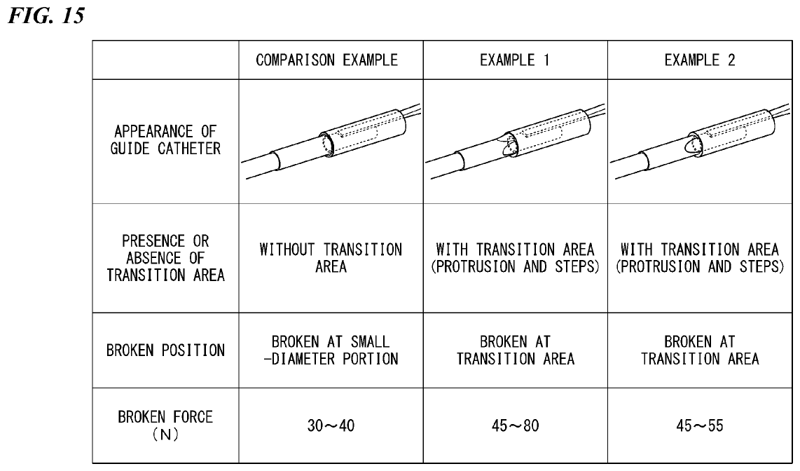

As shown in FIG. 15, the comparison example had a structure in which only the step portion was formed by directly connecting the small-diameter portion and the large-diameter portion. That is, there was no transition region disclosed in the comparison example. In Example 1, the transition region according to the present embodiment having four protrusions was provided. In Example 2, a transition region in which two protrusions having the same shape as that of Example 1 are arranged every 180 degrees in the circumferential direction was provided.

In Example 1 and Example 2, the dimensions of the transition region in the longitudinal direction of the tube were the same with each other.

Thirty tubes of each example were prepared, and the breaking strength and breaking position due to the traction of the wire were examined. The breaking strength indicates the breaking strength when the tube is pulled in the longitudinal direction.

The wire was pulled until the tube broke in the state in which the small-diameter portion was supported by a chuck, and the force at the time of the breaking occurred (breaking force) and the position where the breaking occurred were recorded.

The results are shown in FIG. 15.

In the comparison example, the breaking force of the small-diameter portion at the time of breaking occurred was about 30 to 40 N. On the other hand, in Example 1 and Example 2, the transition portion was broken without the small-diameter portion being broken. The amount of the breaking force at the time of breaking occurred was 45 to 80 N in Example 1 and 45 to 55 N in Example 2.

Accordingly, Example 1 and Example 2 were better than comparison example in that the breaking position was stable at the portion where the breaking force amount was high.

As described above, in the stent delivery device 1 according to the present embodiment, the outer circumferential surface of the transition region 84 provided at the boundary between the small-diameter portion 82 and the large-diameter portion 83 in the guide catheter 80 has the shape to become a partial step portion with respect to the outer circumferential surface of the large-diameter portion 83. As a result, it is possible to stabilize the breaking position at the portion where the breaking strength is high. For example, it is difficult for the guide catheter 80 to be broken even when the guide catheter 80 is operated in the state in which the external force is applied to the distal-end portion (small-diameter portion) of the guide catheter 80.

Furthermore, since the transition region 84 has the recess portion 842, the surface area per unit volume is increased as compared with the truncated-cone-shaped tapered portion without the recess portion 842, and it is possible to make the crystallinity of the transition region to be lower than that of the truncated-cone-shaped tapered portion only by the natural cooling. Therefore, it is possible to make the transition region to be solidified with a low crystallinity without using any special device or the like for quenching the molded tube. As a result, in a case in which a load is applied to the tube 81 in the longitudinal axis direction, the tube 81 is not broken and the transition region 84 is stretched such that it is more likely that the breaking occurs in the transition region 84.

Although one embodiment of the present disclosure has been described above, the technical scope of the present disclosure is not limited to the above-described embodiment. The combination of components may be changed or each component may be changed without departing from the scope of the present disclosure, and various modifications to each configuration elements and deletion may be applied.

Some changes are shown as examples; however, all of the changes are not shown below, and other changes are possible.

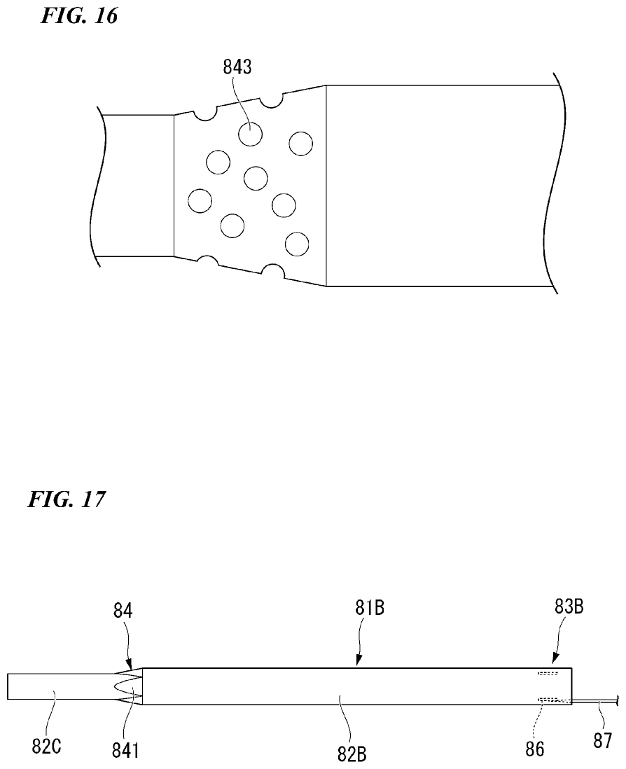

The transition region according to the present disclosure may not have a clear protrusion as long as there is a recess portion configured to increase the surface area per unit volume. For example, as the transition region 84A shown in FIG. 16, as one example of the step portion, a plurality of dimples 843 may be formed on the outer circumferential surface of a conical shape regularly or irregularly. Besides the dimples, linear-shaped or curved (including spiral) grooves may be formed on the outer surface of the transition region as the step portions.

Even in the case in which the protrusions are provided in the transition region, there is no particular restriction on the number, shape, arrangement and the like of the protrusions.

In the above-described embodiment, the transition region is provided between the small-diameter portion through which the stent is passed and the large-diameter portion to which the wire and pipe are attached: however, the portion where the transition region is provided is not limited to this example. For example, in a case of a delivery catheter corresponding to a relatively thick stent, as shown in FIG. 17, in the tube 81B, the portion 82B through which the stent is passed and the portion 83B to which the wire and pipe are attached may have the same diameter. Furthermore, it is possible to form the distal-end portion 82C protruding from the stent to be thinner than these portions, and provide the transition region 84 in these portions. According to this modification example, the portion 82B is treated as the second region and the distal-end portion 82C is treated as the first region. The transition region according to the present disclosure is a portion where the outer diameter gradually decreases from the second region toward the first region in the longitudinal axis direction, and the transition region is suitable for a portion where a relatively large force is applied during the usage. In this case, the stent 10 is attached to the delivery system by inserting the second region as part of the guide catheter 80 inside the stent 10. Also, in the state in which the stent 10 is attached to the delivery catheter 100, the transition region 84 is positioned at the distal end side with respect to the pusher catheter 90 on the hand side.

The transition region may be provided at a plurality of locations of the tube.

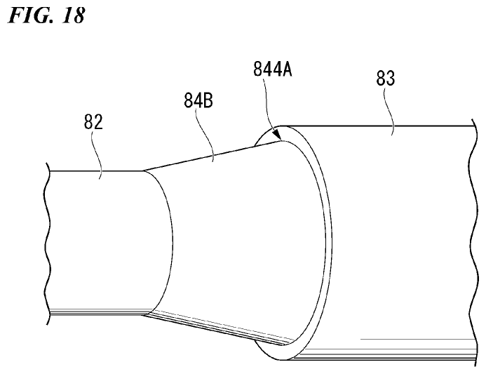

As in the modification example as shown in FIG. 18, the proximal end of the transition region 84B may have a step portion 844A over the entire circumference with respect to the outer circumferential surface of the large-diameter portion 83. More specifically, the transition region 84B may be formed in a truncated cone shape, and a step portion 844A may be formed over the entire circumference at the boundary with the large-diameter portion 83 (the proximal end of the transition region 84B). Even with such a configuration, by decreasing the crystallinity of the transition region 84B in the catheter manufacturing process, when a load is applied to the tube in the longitudinal axis direction, it is easier for the transition portion 84B to be stretched while the small-diameter portion 82 is not broken such that the breaking is more likely to occur in the part of the step portion 844A at the boundary between the transition region 84B and the large-diameter portion 83 and it is possible to achieve two goals of realizing the high breaking strength and stabilizing the breaking position at the same time.

The medical catheter to which the present disclosure is applied is not limited to the above-described delivery catheter of the stent. If the catheter has a portion where the outer diameter varies, the above-described transition region can be formed without restrictions on the application, structure, or the like so as to increase the breaking strength while stabilizing the breaking position.

The present disclosure includes the following contents.

A manufacture method of a catheter, comprising:

disposing a material tube in a mold having a second cavity with a second inner diameter and a transition cavity having a convex portion on an inner surface and communicating with the second cavity, wherein an inner diameter of the transition cavity decreases as the transition cavity separates from the second cavity;

melting the material tube in the mold so as to fill the second cavity and the transition cavity; and

cooling the material tube in the mold to form the catheter having a second region with a second outer diameter corresponding to the second inner diameter, a transition region having a shape corresponding to the transition cavity, and a first region connecting with the transition region and having a first outer diameter smaller than the second outer diameter.

Although the respective embodiments and modifications of the present disclosure have been described above, the technical scope of the present disclosure is not limited to the above-described embodiments, and configurations in the respective embodiments and modifications within the scope not departing from the spirit of the present disclosure. It is possible to change the combination of elements, make various changes to each configuration element, or delete each configuration element. For example, the configuration according to any one of above-described embodiments and modifications of the present disclosure may be appropriately combined with each modification of the operation portion. The present disclosure is not limited by the above description, but only by the appended claims.

What is claimed is:

1. A catheter, comprising:
a first region having a first outer diameter;
a second region having a second outer diameter larger than the first outer diameter; and
a transition region connecting with the first region and the second region, the transition region having an outer diameter being equal to or larger than the first outer diameter and equal to or smaller than the second outer diameter,
wherein the outer diameter of the transition region gradually decreases from the second region toward the first region, and
part of an outer circumferential surface of the transition region is formed in a shape having a step portion.

2. The catheter according to claim 1,
wherein the step portion is a recess portion formed on the outer circumferential surface of the transition region, and
a diameter-direction dimension of the recess portion is smaller than the second outer diameter.

3. The catheter according to claim 2,
wherein the transition portion has a plurality of protrusions, and
the recess portion is positioned between the plurality of protrusions.

4. The catheter according to claim 3,
wherein the plurality of protrusions are arranged in a circumferential direction of the catheter at equal intervals therebetween.

5. The catheter according to claim 1, further comprises:
a pipe arranged coaxially with respect to the catheter by being embedded in the second region; and
a wire connected with the pipe.

6. The catheter according to claim 1, wherein the step portion is formed in a concave shape in the circumferential direction of the transition region.

7. The catheter according to claim 1, wherein the step portion is formed in a proximal end of the transition region along a longitudinal direction of the transition region.

8. The catheter according to claim 1, wherein a breaking strength of the transition region along a longitudinal direction of the transition region is in a range from 45N to 80N.

9. A stent delivery device, comprising:
a delivery catheter including a tubular stent and a guide tube inserted into the stent,
wherein the guide tube comprises:
a first region having a first outer diameter;
a second region having a second outer diameter larger than the first outer diameter; and
a transition region connecting with the first region and the second region, the transition region having an outer diameter being equal to or larger than the first outer diameter and equal to or smaller than the second outer diameter,
wherein the outer diameter of the transition region gradually decreases from the second region toward the first region,
part of an outer circumferential surface of the transition region is formed in a shape having a step portion, and
the step portion is positioned between a proximal end of the stent and the second region.

10. The stent delivery device according to claim 9,
wherein the step portion is a recess portion formed on the outer circumferential surface of the transition region, and
a diameter-direction dimension of the recess portion is smaller than the second outer diameter.

11. The catheter according to claim 10,
wherein the transition portion has a plurality of protrusions, and
the recess portion is positioned between the plurality of protrusions.

12. The catheter according to claim 11,
wherein the plurality of protrusions are arranged in a circumferential direction of the catheter at equal intervals therebetween.

13. A stent delivery device, comprising:
a delivery catheter including a tubular stent and a guide tube inserted into the stent,
wherein the guide tube comprises:
a first region having a first outer diameter;
a second region having a second outer diameter larger than the first outer diameter; and
a transition region connecting with the first region and the second region, the transition region having an outer diameter being equal to or larger than the first outer diameter and equal to or smaller than the second outer diameter, and
wherein a breaking strength of the transition region along a longitudinal direction of the guide tube is in a range from 45N to 80N.
